(19) United States
(12) Patent Application Publication
(10) Pub. No.: US 2009/0182207 A1
(43) Pub. Date: Jul. 16, 2009

(54) INGESTIBLE ANIMAL HEALTH SENSOR

(75) Inventors: Frank Riskey, Boise, ID (US); Shawn Flagstad, Emmett, ID (US); Nathan D. Riskey, Boise, ID (US); Petru Sandor Jr., Nampa, ID (US)

Correspondence Address: STOEL RIVES LLP - SLC, 201 SOUTH MAIN STREET, SUITE 1100, ONE UTAH CENTER, SALT LAKE CITY, UT, 84111, US

(73) Assignee: TENXSYS INC., Eagle, ID (US)

(21) Appl. No.: 12/015,163

(22) Filed: Jan. 16, 2008

(51) Int. Cl. A61B 5/07; A61B 5/103

(52) U.S. Cl. 600/302; 600/587

(57) ABSTRACT

An ingestible bolus configured to be maintained in a stomach of an animal may be formed from a substantially cylindrical enclosure shell. Ballast weight and a power source may be disposed therein. The ballast weight may be configured to cause the bolus to be maintained in contact with a stomach wall of the animal when disposed therein. The bolus may comprise sensors to measure one or more animal characteristics, a transmitter in wireless communication with an animal monitoring system, a memory unit to store measured characteristics and bolus configuration data, and processor to control the operation of the bolus components. The transmitter may be in communication with an antenna, which may be formed as a trace on an antenna circuit substrate, such as a printed circuit board (PCB). The antenna may be separated from the sensors, processors, and transmitter to prevent and/or reduce interference and/or coupling therebetween. The accelerometer sensor may be a three-axis accelerometer and may be configured to detect animal movement characteristics and animal stomach contractions.

### TECHNICAL FIELD

This disclosure relates generally to a system and method for monitoring one or more animal characteristics and, in particular, to an ingestible bolus disposable within a lower portion of an animal's stomach to measure and wirelessly transmit one or more measured animal characteristics therefrom.

### BRIEF DESCRIPTION OF THE DRAWINGS

The various aspects and advantages of the invention are described by way of example in the following description of several embodiments and attached drawings. It should be understood that the accompanying drawings depict only typical embodiments and, as such, should not to be considered to limit the scope of the claims. The embodiments will be described and explained with specificity and detail in reference to the accompanying drawings in which:

FIG. 1 is a block diagram of one embodiment of a system for monitoring an animal;

FIG. 2 is a block diagram of a one embodiment of an ingestible bolus;

FIG. 3 is a diagram of one embodiment of an ingestible bolus;

FIG. 4 is a diagram of one embodiment of a substantially disk-shaped printed circuit board substrate; and

FIG. 5 is a diagram of one embodiment of a substantially disk-shaped antenna disposed on a printed circuit board substrate.

### DETAILED DESCRIPTION OF PREFERRED EMBODIMENTS

This disclosure relates generally to systems and methods for monitoring a health condition of an animal and, in particular, to systems and methods for monitoring one or more animal characteristics to detect a health condition of the animal.

It will be readily understood that the components of the present invention, as generally described and illustrated in the Figures herein, could be arranged and designed in a wide variety of different configurations. Thus, the following detailed description of the embodiments of the apparatus, system, and method of the disclosure is not intended to limit the scope of the disclosure, as claimed, but is merely representative of possible embodiments of the disclosure.

In some cases, well-known structures, materials, or operations are not shown or described in detail. Furthermore, the described features, structures, or characteristics may be combined in any suitable manner in one or more embodiments. It will also be readily understood that the components of the embodiments as generally described and illustrated in the Figures herein could be arranged and designed in a wide variety of different configurations.

The order of the steps or actions of the methods described in connection with the embodiments disclosed may be changed as would be apparent to those skilled in the art. Thus, any order in the Figures or description is for illustrative purposes only and is not meant to imply a required order, unless specified to require an order.

Certain aspects of the embodiments described may be illustrated as hardware components, or software modules or components. As used herein, a software module or component may include any type of computer instruction or computer executable code located within a memory device and/or transmitted as electronic signals over a system bus or wired or wireless network. A software module may, for instance, comprise one or more physical or logical blocks of computer instructions, which may be organized as a routine, program, object, component, data structure, etc., that perform one or more tasks or implements particular abstract data types. In certain embodiments, a particular software module may comprise disparate instructions stored in different locations of a memory device, which together implement the described functionality of the module. Indeed, a module may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices.

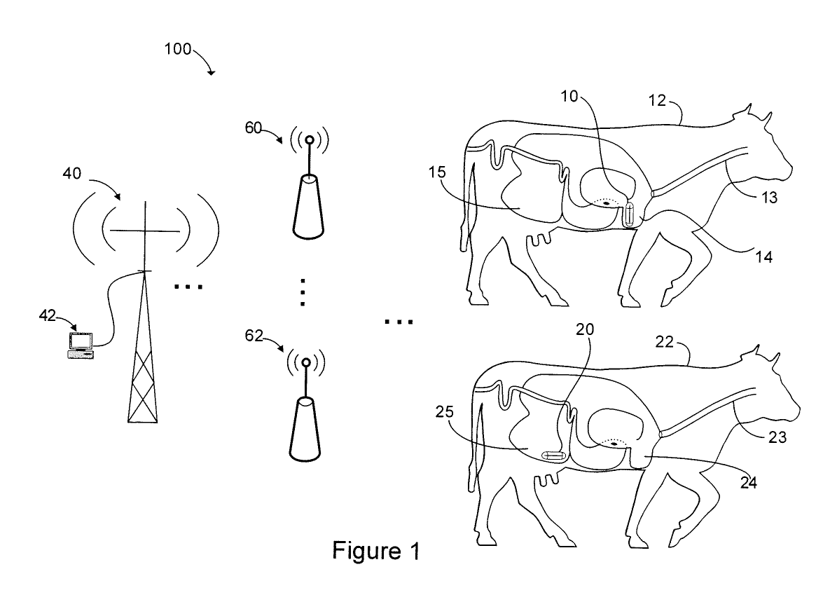

Turning now to FIG. 1, a block diagram 100 of one embodiment of a system for monitoring one or more animal characteristics is depicted. In FIG. 1, an ingestible bolus 10, 20 may be disposed within the body of an animal 12, 22. Bolus 10, 20 may be configured to be ingested via the esophagus 13, 23 of a ruminant animal 12, 22, such as a bovine. In this embodiment, bolus 10, 20 may be configured to have a size and density which will enable it to remain within the stomach of a bovine 12, 22, ensuring that it is not regurgitated from the animal's reticulum 14, 24 and/or rumen 15, 25.

FIG. 1 shows ingestible bolus 10 within reticulum 14 of ruminant animal 12 and ingestible bolus 20 within rumen 24 of ruminant animal 22. Ingestible bolus 10, 20 may be maintained in any stomach and/or stomach chamber capable of holding bolus 10, 20. Bolus 10, 20 may be capable of remaining in the animal's reticulum 14, 24 and/or rumen 15, 25 throughout the life of the animal 12, 22.

In an alternative embodiment, bolus 10, 20 may be injected into and/or implanted under the skin of an animal 12, 22 or otherwise implanted within the body of an animal 12, 22.

Bolus 10, 20 may comprise wireless communications means to allow bolus 10 to be in wireless communication with base station 40. Base station 40 may comprise a computing device 42 communicatively coupled to base station 40. Computing device 42 may be any general and/or specific purpose computing device known in the art.

The base station 40 and/or computing device 42 may be monitored by and/or in communication with an animal manager. As used herein, an animal manager may refer to any person, machine, and/or process used to manage one or more animals. The animal manager may be in charge of and/or responsible for one or more animals monitored by the systems and methods disclosed herein. An animal manager may comprise one or more automated systems, such as feeding, heating, cooling and other systems. An animal manager may further comprise a human animal manager and/or veterinarian. Base station 40 and/or computing system 42 may be configured by and/or interact with the animal manager to monitor and/or respond to changes in animal 12, 22 health condition.

The wireless communication means of bolus 10, 20 may comprise a wireless transmitter and/or receiver operating at 900 MHz or some other suitable radio frequency (RF). In some embodiments, such wireless communication may be two-way, allowing bolus 10, 20 to transmit and receive data from base station 40. In other embodiments, bolus 10, 20 may only be capable of transmitting data to base station 40.

Animals 12 and 22 may be capable of roaming over a relatively large area, such as a feed lot, dairy, range area, enclosure, or the like. As such, the distance between base station 40 and bolus 10, 20 may become greater than the wireless communication range of bolus 10, 20. In this case, one or more wireless transponders 60, 62 may be deployed to increase the communications range of bolus 10, 20 to base station 40. Transponder(s) 60, 62 may receive wireless transmissions from bolus 10 and retransmit them at a higher power and/or a different frequency to allow such transmissions to be received by base station 40. Similarly, in this embodiment, transponder(s) 60, 62 may receive transmissions from base station 40 to bolus 10 and retransmit them at higher power so that they may be received by bolus 10, 20.

Multiple wireless transponders 60, 62 may be disposed within the vicinity of animals 12, 22. For example, FIG. 1 depicts two wireless transponders 60, 62 that may be in wireless communication with bolus 10, 20. Where there are multiple base stations 40 and/or transponders 60, 62 within the wireless communication range of bolus 10, 20, boluses 10, 20 may be configured to communicate with only the base station 40 and/or transponder 60, 62 with the strongest communications signal. In this embodiment, each base station 40 and transponder 60, 62 may be configured to communicate on a separate frequency and/or channel within an RF frequency range (e.g., 900 MHz). The bolus 10, 20 may be configured to receive data on each of the communications channels used by base station 40 and transponders 60, 62 to listen for the strongest signal received on each. Upon determining the best (e.g., strongest signal and/or least loaded) wireless communication channel (e.g., transponder 60, 62 and/or base station 40), bolus 10, 20 may transmit and/or receive data using only that channel. Bolus 10, 20 may be configured to periodically re-evaluate the available channels to allow bolus 10, 20 to adapt to changes in animal position and/or changes in base station 40 and/or transponder 60, 62 signal strength and/or configuration. Similarly, if bolus 10, is out of communication range with any of transponder(s) 60, 62 and base station 40, bolus 10, 20 may be configured to cease transmission and/or only transmit transponder 60, 62 and/or base station 40 discovery messages.

Although FIG. 1 depicts two boluses 10 and 20 and two transponders 60 and 62, one skilled in the art would recognize that an animal monitoring system according to the teachings of this disclosure could comprise a virtually unlimited number of transponders 60, 62 animals 12, 22, and/or boluses 10, 20. As such, this disclosure should not be read as limited to any particular transponder 60, 62, animal 12, 22, and/or bolus 10, 20 configurations.

Bolus 10, 20 may comprise one or more sensors to detect one or more characteristics of animal 12, 22. In this embodiment, bolus 10, 20 may wirelessly transmit data corresponding to monitored animal characteristics to base station 40. Monitored animal characteristics may include physiological characteristics, such as animal temperature, stomach pH, blood pH, heart rate, respiration, stomach contractions, and the like. Monitored animal characteristics may also include non-physiological characteristics, such as animal movement and/or motion activity, animal location, and the like.

The wireless communication characteristics of bolus 10, 20 between base station 40 and/or transponder(s) 60, 62 may allow base station 40 to determine location information relating to animal 12, 22. In an embodiment employing only a single base station 40, base station 40 may be configured to determine the animal's 12, 22 distance from base station 40. This may be done in a variety of ways including, but not limited to: determining distance based upon wireless signal strength; determining distance from timestamp information in the wireless message; or the like.

As shown in FIG. 1, the system may comprise more than one base station 40 and/or transponder(s) 60, 62. In this case, base station 40 may determine location information relating to animal 12, 22 using well-known wireless communications triangulation methods.

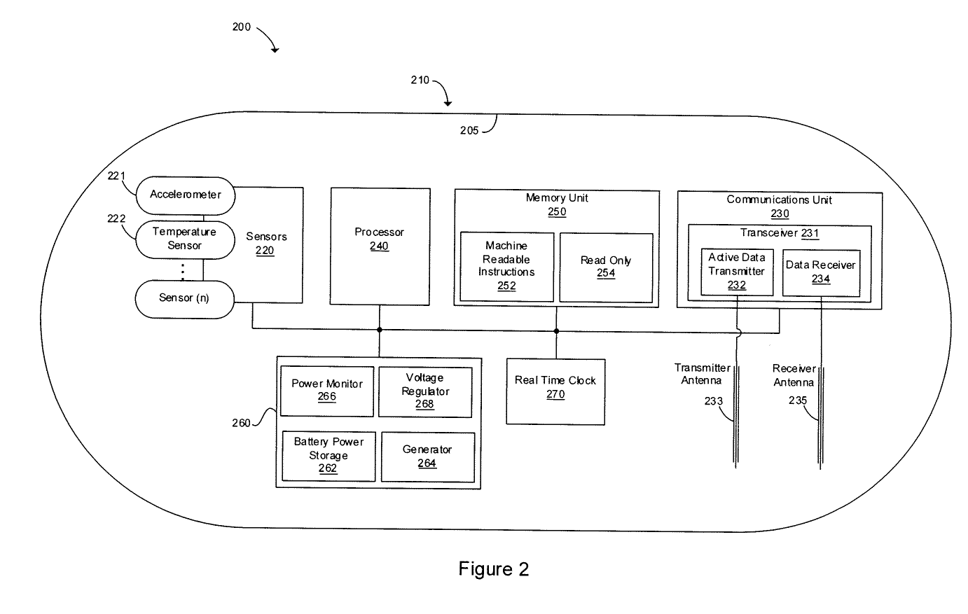

Turning now to FIG. 2, a block diagram of one embodiment 200 of a bolus 210 is depicted. The components of bolus 210 may be disposed within an enclosure 205. Enclosure 205 may be formed from any material capable of remaining within the stomach of an animal without deteriorating and/or degrading. In addition, enclosure 205 may be formed of a material unlikely to produce an adverse reaction in an animal when implanted therein (e.g., within the rumen or reticulum of the animal). In one embodiment, enclosure 205 may be formed from a non-toxic plastic material.

Bolus 210 may comprise one or more sensors 220 configured to measure one or more animal characteristics. One or more of sensors 220 may detect animal movement and/or motion activity characteristics including, but not limited to: distance traveled by the animal; animal movement frequency; animal movement speed; and the like. In one embodiment, an accelerometer 221 may be used to detect such movement and/or motion activity characteristics. In this embodiment, accelerometer 221 may be a three-axis accelerometer capable of detecting animal movement and/or motion activity in each of the Cartesian “x,” “y,” and “z” axes. Bolus 210 may change its orientation while within an animal (e.g., within an animal's rumen and/or reticulum). As such, detection of movement and/or motion activity in only one (1) or two (2) axes may yield inaccurate results.

An acceleration vector magnitude (VM) value may be calculated from the readings of the three-axis accelerometer by calculating a square root of the sum of squares of each of the “x,” “y,” and “z” coordinate axes as shown in Equation 1.1:

VM=\sqrt{x²+y²+Z²} Eq. 1.1

A derivative of the vector magnitude (VM) may be approximated by calculating the absolute value of the difference between subsequent vector magnitude values as illustrated in equation 1.2:

$$\frac{\partial {VM}_{n}}{\partial t} = \vert {VM}_{n} - {VM}_{n - 1} \vert \qquad Eq . 1.2$$

The derivative of acceleration calculated per equation 1.2 may be useful in monitoring animal characteristics as it may remove errors caused by “sloshing” movement of bolus 210 within the stomach of the animal or other constant acceleration forces acting on the animal (e.g., gravity). Accordingly, the derivative of the movement and/or motion activity vector magnitude may provide an accurate representation of the actual movement and/or motion activity characteristics of the animal.

In addition to detecting movement and/or motion activity, accelerometer 221 may be configured to detect stomach contractions. Since bolus 210 may be disposed in the stomach of an animal (e.g., in a ruminant animal's rumen or reticulum), bolus 210 may be movably affected by animal stomach contractions. In this case, bolus 210 may be configured to have a weight and/or density sufficient to cause bolus 210 to be maintained in a lower portion of the animal stomach and/or be movably coupled to a wall of the animal's stomach. As used herein, a bolus 210 movably coupled and/or in movable communication with a stomach of an animal may refer to a condition wherein a movement of the stomach, such as a contraction, causes a corresponding movement detectable by accelerometer 221 the bolus 210. As such, bolus 210 may be movably coupled and/or in movable communication with a stomach wall, but not in contact with the stomach wall (e.g., bolus 210 may be movably coupled to the stomach wall via other materials in the stomach, such as feed material and/or fluids within the stomach). Stomach contraction movement may be detected by accelerometer 221 as a periodic acceleration. For example, a typical ruminant animal may experience a three (3) stomach contractions every minute while “on-feed” (i.e., every 20 seconds). As used herein, “on-feed” may refer to an animal health condition wherein the animal is actively feeding, and “off-feed” may refer to an animal health condition wherein the animal is no longer and/or intermittently feeding. Accordingly, an animal manager may be able to detect when an animal goes “off-feed” by observing a change and/or reduction in the frequency of animal stomach contractions. This may be important since going “off-feed” may be indicative of a more serious health condition. In addition, an animal may go “off-feed” before other health-condition symptoms (e.g., increased/decreased movement, temperature, or the like) are observable.

In one embodiment, bolus 210 may comprise one or more sensors 220 capable of determining the position of bolus 210, such as a Global Positioning System (GPS) receiver or the like. In this embodiment, a GPS receiver may be used to detect animal position, and animal movement, and/or animal motion activity characterist

One or more sensors 220 of bolus 210 may be used to detect physiological characteristics of an animal including, but not limited to: body temperature; heart rate; respiration; stomach contractions; stomach pH; blood pH; and the like. Any number of sensors 220 may be used to detect such characteristics. For example, to detect animal temperature, a temperature sensor 222 may be employed. Temperature sensor 222 may comprise a thermistor, thermocouples or a platinum resistance thermometer, or the like.

It would be understood by one skilled in the art that any number of sensors 220 could be included within bolus 210 under the teachings of this disclosure. As such, this disclosure should not be construed as limited to any particular sensors 220.

In one embodiment, bolus 210 may comprise a wireless communication module 230. Communication module 230 may comprise transceiver 231. Transceiver 231 may be capable of transmitting and receiving data via a single antenna (not shown) or a separate transmitter antenna 233 and receiver antenna 235. As such, in one embodiment, transceiver 231 may comprise active data transmitter 232 and data receiver 234. Active data transmitter 232 may be communicatively coupled to transmitter antenna 233. Transmitter antenna 233 may be disposed within enclosure 205 of bolus 210, upon the surface thereof, or may be disposed externally to enclosure 205 of bolus 210. Data receiver 234 may be communicatively coupled to receiver antenna 235. Receiver antenna 235 may be disposed within enclosure 210 of bolus 10, upon the surface thereof, or may be disposed externally to enclosure 210 of bolus 10. In one embodiment, transmitter antenna 233 may be capable of transmitting data at 900 MHz, and receiver antenna 235 may be capable of receiving data at 900 MHz or some other suitable frequency. In another embodiment, transmitter antenna 233 and receiver antenna 235 may be comprised of a single antenna (not shown) used for both data transmission and reception.

Bolus 210 may comprise a processor 240 communicatively coupled to a memory unit 250. In one embodiment, memory unit 250 may comprise machine readable instructions 252 stored thereon. In this embodiment, processor 240 may read and execute machine readable instructions 252 stored on memory unit 250. Machine readable instructions 252 may comprise bolus configuration data, which may include, but is not limited to: a polling and/or sampling frequency of one or more of sensors 220, a transmission frequency of communications unit 230; calibration information for one or more of sensors 220; or the like.

Processor 240 may be communicatively coupled to each of sensors 220. Machine readable instructions 252 stored on memory unit 250 may specify a sensor sampling frequency for each of the sensors 220. As used herein, a sensor sampling frequency may determine how often a sensor reading is obtained from a particular sensor 220. For example, a sensor sampling frequency may define how often temperature sensor 222 obtains a temperature sensor reading or sensor sample from the animal. The processor 240 may configure one or more of sensors 220 with a sensor sampling frequency specified by machine readable instructions 252. Alternatively, one or more sensors 220 may be communicatively coupled to memory unit 250 and may be configured to read a sensor sampling frequency directly from the machine readable instructions 252.

Machine readable instructions 252 may specify a sensor reading duration for each of sensors 220. As used herein, a sensor reading duration may define the length of time a particular sensor 220 may obtain a reading. For example, a reading duration may define how long accelerometer 221 reads animal movement and/or motion activity characteristics. A reading duration may specify that accelerometer 221 should read animal movement and/or motion activity characteristics for one minute each time a sensor sample is taken. Processor 240 may configure one or more of sensors 220 with a sensor reading duration specified by machine readable instructions 252. Alternatively, one or more sensors 220 may be communicatively coupled to memory unit 250 and may be configured to read their sensor reading duration directly from machine readable instructions 252.

Machine readable instructions 252 may specify calibration information for one or more sensors 220. In this embodiment, one or more sensors 220 may be tested to determine whether accurate readings are being returned. In the event a particular sensor 220 is not returning accurate readings, calibration data may be stored within memory unit 250 to rectify the readings to a correct value. In this embodiment, sensor 220 may be communicatively coupled to memory unit 250 to allow a sensor 220 to read the calibration data therefrom. Sensor 220 may itself comprise a memory storage location whereon such calibration information may be stored. Machine readable instructions 252 may instruct processor 240 to transfer sensor calibration data stored within memory unit 250 to the memory storage location of a particular sensor 220. In another embodiment, sensor 220 may not comprise a memory storage location and may not be capable of reading memory unit 250. As such, machine readable instructions 252 may configure processor 240 to apply calibration data stored within memory unit 250 to readings returned by sensors 220.

Machine readable instructions 252 may specify that one or more sensors 220 should be deactivated in order to reduce the power consumed by bolus 210. Processor 240 may be communicatively coupled to sensors 220 and may be capable of configuring and/or controlling one or more of sensors 220. Machine readable instructions 252 may specify that one or more sensors 220 should be re-activated.

Processor 240 may be communicatively coupled to sensors 220 and may control the operation and configuration of sensors 220. Processor 240 may poll one or more of sensors 220 at a polling interval (i.e., polling frequency) specified by machine readable instructions 252 stored in memory unit 250. As used herein, polling a sensor refers to obtaining measurement data from one or more sensor 220. Polling a sensor may comprise processor 240 sending a query to a sensor 220, and responsive to this query, sensor 220 may obtain and return to processor 240 the sensor reading. For example, temperature sensor 222 may respond to polling by reading and returning the current animal temperature. In another embodiment, polling a sensor may comprise causing processor 240 to read the current sensor value from a sensor 220. In another embodiment, one or more sensors 220 may be configured to store sensor measurements on memory unit 250. One or more sensors 220 may be configured with a sensor sampling frequency that is greater than the polling frequency of processor 240. As such, sensors 220 may store multiple sensor samplings on memory unit 250 between polling intervals of processor 240. Accordingly, polling a sensor 220 may comprise processor 240 reading all of the sensor readings stored on memory unit 250 for each of the one or more sensors 220.

In another embodiment, sensor 220 may alternatively comprise a memory storage location to store sensor samples. In this embodiment, processor 240 may poll sensor 220 by reading a sensor 220 storage location. In another embodiment, sensor 220 may have a sensor reading duration to allow sensor 220 to measure animal characteristics over time (e.g., an accelerometer sensor 221). Sensor 220 may store such measurements on an internal sensor storage location or on memory unit 250. The processor 240 may poll such a sensor by reading memory 250 or the internal storage location of the sensor 220. In this embodiment, processor 240 may be configured to pre-processes the measurement data before transmission. Such preprocessing may comprise calculating a measurement characteristics, such as mean, standard deviation, etc., compressing the data, and the like. As such, the preprocessing may reduce the amount of data transmitted to the base station (not shown) and thereby reduce power and RF transmission requirements.

In addition to transmitting animal characteristics obtained by one or more bolus 210 sensors 220, bolus 210 may be configured to transmit bolus 210 status information including, but not limited to: power available in power source 260 (e.g., monitored by power monitor 266); status of sensors 220, processor 240 and/or memory unit 250; or the like. A base station (not shown) may use this status information to alert an animal manager to a possible problem with the bolus 210 (e.g., power source 260 about to expire, etc.).

It should be understood that bolus 210 may comprise sensors 220 having any number of sampling or measurement storage techniques and that processor 240 may be configured by machine readable instructions 252 to poll sensors 220 having such various sampling or measurement storage techniques.

Machine readable instructions 252 may specify a polling frequency for each sensor 220 or may specify a common polling interval for all or a sub-set of sensors 220. As used herein, a polling frequency may specify how often processor 240 polls one or more sensors 220.

In one embodiment, machine readable instructions 252 may define conditions under which the polling frequency associated with one or more sensors 220 may change. For example, machine readable instructions 252 may instruct processor 240 to increase the polling frequency and/or sensor sampling frequency of a temperature sensor 222 in the event that the animal temperature exceeds a threshold value. Instructions 252 may instruct processor 240 to decrease the polling frequency and/or sensor sampling frequency of the temperature sensor 222 if the animal temperature is maintained below a threshold value. Processor 240 may adapt the polling frequency and/or sensor sampling frequency to changing animal health conditions so that potential health risks and/or other changes in animal health state may be recognized as soon as possible while minimizing extraneous sensor measurements and message transmissions. The frequency of sensor 220 polling transmission may be configured to change according to animal location. For example, the processor 240 may increase a polling and/or transmission frequency when the animal is in the vicinity of a calving pen and/or hospital pen, and may decrease the polling and/or transmission frequency when the animal is moved out of the pen. Similarly, the sensor polling and/or transmission frequencies may be configured to vary based upon an animal schedule. For example, bolus 210 may be configured to transmit measurements during animal milking time (e.g., three (3) times per day). Time information for such time-dependent instructions 252 may be provided by real time clock 270, which is discussed in more detail below.

In one embodiment, processor 240 may transmit sensor measurements obtained by polling sensors 220 via data transmitter 232. In one mode of operation, processor 240 may form a message comprising the measurements as sensor 220 readings are obtained (after polling the one or more sensors 220). Such a message may be referred to as an animal characteristics message and may be comprised of the sensor readings obtained by polling one or more sensors 220. This operational mode may be referred to as “instantaneous” mode since sensor readings are transmitted as they are polled by processor 240. In another mode of operation, processor 240 may not immediately transmit the sensor readings polled from sensors 220, but instead store them on memory unit 250. In this mode, machine readable instructions 252 may specify a transmission internal, wherein processor 240 may transmit an animal characteristics message comprising some or all of the measurements stored in memory unit 250 at each transmission interval. This operational mode may be referred to as “burst” mode since sensor 220 readings are transmitted as periodic bursts rather than when sensor polling takes place. Operation in “burst” mode may reduce the power consumed by bolus 210 by reducing the number of transmissions sent from data transmitter 232.

In one embodiment, messages transmitted via data transmitter 232 of communication module 230 may comprise a media access control (MAC) value. A MAC may be a six (6) or three (3) byte value used to uniquely identify messages originating from a particular bolus 210. A MAC address may also be used by data receiver 234 and/or processor 240 to identify messages intended for bolus 210. As such, receiver 234 and/or processor 240 may disregard any incoming messages having a MAC address other than its own, obviating the need to time-slice or otherwise manage wireless traffic between bolus 210 and a base station or other wireless device. MAC addressing to route and control network messages is generally known within the networking arts.

In one embodiment, a programmable unique animal identifier (UAID) may be stored on memory unit 250. In this embodiment, the UAID may be used to associate a bolus 210 with a particular animal. The UAID value may be transmitted with some or all of the messages originating from a particular bolus 210, allowing the receiver of such messages to associate the received data with a particular animal.

In one embodiment, the bolus memory unit 250 may comprise read-only storage 254. Read-only storage 254 may be a Programmable Read-Only Memory (PROM), Erasable Programmable Read-Only Memory (EPROM), Electrically Erasable Programmable Read-Only Memory (EEPROM), or the like. In this embodiment, a unique bolus identifier value (UBID) may be stored within the read-only storage 254. The UBID value may be transmitted with some of all of the messages transmitted from the bolus 210. In this embodiment, the UBID may provide a tamper-proof identifier to uniquely identify a particular bolus 210. As used herein, an “animal identifier” or “bolus identifier” may refer to any of the above mentioned animal and/or bolus identifier values.

In one embodiment, communication module 230 may detect whether bolus 210 is within range of a receiver, such as a base station (not shown) or transceiver (not shown). Processor 240 may cause communication module 230 to transmit a simple message at a set interval. This simple message may be referred to as a “ping” and may include one or more of the unique identifiers associated with a particular bolus 10 (e.g., a MAC, UAID, and/or UBID). A base station or transceiver receiving the ping message may be configured to send a short reply message indicating that the ping message was received. In this way, processor 240 may know that it is within wireless range of a base station or transceiver. Upon receipt of a reply message, bolus 210 may be configured to be in “on-line” mode. If bolus 210 does not receive a reply message within a threshold period of time, it may transmit additional ping messages. If a threshold number of retry ping messages have been sent without a reply, bolus 210 may be configured to be in “off-line” mode. Machine readable instructions 252 may include instructions to be executed by processor 240 corresponding to “on-line” and/or “off-line” mode.

Alternatively, communication unit 230 may be configured to listen for, rather than transmit, the ping discovery messages discussed above. In this embodiment, a base station (e.g., base station 40 of FIG. 1), may be configured to transmit periodic ping messages. Upon receiving one of these messages through communication unit 230, processor 240 may cause bolus 210 operate in “on-line” mode and, if no ping messages are received for a threshold time period, processor 240 may cause bolus 210 to operate in “off-line mode.” This may allow bolus 210 to conserve power and may reduce RF interference potentially caused by transmitting periodic ping messages.

In “on-line” mode, bolus 210 may transmit animal characteristics messages at the “online” transmission frequency specified by machine readable instructions 252. As discussed above, such messages may be transmitted as processor 240 polls sensors 220, or may be transmitted at a periodic transmission interval. The receiver of such messages may be configured to respond with a confirmation message. The confirmation message may be used in the place of a separate ping message in order to decrease the message traffic between bolus 210 and the receiver.

In “off-line” mode, bolus 210 may decrease transmission frequency of messages according to machine readable instructions 252. Additionally, while in “off-line” mode, machine readable instructions 252 may direct processor 240 to deactivate certain sensors 220 in order to conserve power. In “off-line” mode, bolus 210 may continue sending “ping” messages in order to discover when bolus 210 comes back into range of a base station or transceiver unit. In this sense, data transmitter 232 of bolus 210 may be considered to be an active transmitter since bolus 210 may actively transmit animal characteristic messages and may actively detect when a base station or transceiver is in wireless communications range. Bolus 210 may actively transmit animal characteristics and/or detect wireless communications without requiring interrogation by an external source.

In one embodiment, bolus 210 may receive new and/or modified machine readable instructions 252 via data receiver 234 of wireless communication module 230. Such received instructions may comprise changes to the operation of sensors 220 and/or processor 240 including: sensor sampling frequency; sensor reading duration; sensor activation status; sensor calibration data; processor polling frequency; processor operational mode (i.e., “instantaneous” or “burst); and the like.

The embodiment of FIG. 2 may comprise power source 260 coupled to each of sensors 220, communication module 230 including data transmitter 232 and data receiver 234, processor 240, memory unit 250, and any other power-consuming component of bolus 210. Power source 260 may comprise a battery energy storage device 262, such as a lithium ion battery, lead acid battery, nickel cadmium battery, or the like. In another embodiment, power source 260 may comprise a generator 264. Generator 264 may comprise a piezoelectric generator or mass/alternator generator to generate power from the movement and/or motion activity or vibration of bolus 210 within a host animal. In another embodiment, generator 264 may be a heat-activated generator to generate electrical energy from the body heat of a host animal. Generator 264 may comprise a Microbial Fuel Cell (MFC) configured to generate electrical energy derived from bacteria feeding on organic material within an animal's stomach (i.e., rumen or reticulum). Generator 264 may be disposed outside of the bolus enclosure 205. Power source 260 may comprise both battery power storage 262 and generator 264; in this embodiment, power generated by power generator 264 may be stored in battery power storage 262.

Power source 260 may comprise a power monitor 266, which may be used to monitor a power and/or voltage level of battery power storage 262 and/or generator 264. Power monitor 266 may provide power status information to processor 240 and/or communications unit 230. Communications unit 230 may transmit power status information to a base station (not shown), and processor 240 may use power status information provided by power monitor 266 to invoke one or more power-save operations. For example, power monitor 266 may detect a low power condition in power storage 260 (e.g., in batter power storage 262 and/or generator 264). Responsive to the detection, processor 240 may invoke one or more power-save operations defined by machine readable instructions 252 of memory unit 250, which may include, but are not limited to: reducing a polling and/or transmission frequency of bolus 210; deactivating one or more of sensors 22; or the like.

Power source 260 may comprise voltage regulator 268. Voltage regulator 268 may be configured to maintain a steady input voltage level for the components of bolus 210 (e.g., sensors 220, communications unit 230, processor 240, memory unit 250, etc.). Voltage regulator 268 may be capable of maintaining a relatively constant voltage despite fluctuations in the voltage level produced by battery power storage 262 and/or generator 264. Voltage regulator 268 may comprise any voltage regulator implementation known in the art including, but not limited to: a shunt regulator such as a zener diode, avalanche breakdown diode, or voltage regulator tube; a linear regulator; switching regulator; silicon controlled regulator (SRC); a hybrid regulator; or the like.

Bolus 210 may further comprise a real time clock 270 communicatively coupled to processor 240. Real time clock 270 may be used to supply time information to processor 240. This time information may be used to perform one or more time-dependent operations defined by machine readable instructions 252. For instance, some monitoring tasks, such as movement monitoring, may be time-dependent (e.g., movement may not be monitored while an animal is asleep or resting). As such, animal movement sensors may be deactivated at certain times. In addition, machine readable instructions 252 may indicate that the bolus 210 should shut down at certain times in order to conserve power, transmit animal characteristics at certain times, or the like. Time information from real time clock 270 may allow processor 240 to properly execute such time-dependent instructions 252.

Referring again to FIG. 1, one or more bolus 10, 20 may be in wireless communication with a base station 40. As such, bolus 10, 20 may periodically transmit animal characteristics messages to the base station 40. Base station 40 may be communicatively coupled to and/or may comprise a general and/or special purpose computing device 42. This computing device 42 may be configured to create one or more animal profiles associated with one or more animals 12, 22. The device may be further configured to compare any animal characteristics messages to one or more stored animal profiles. As a result of this comparison, the computing device 42 may modify the configuration of the bolus 10, 20 (e.g., modify the bolus 10, 20 polling frequency, sample time, transmission time or the like), may detect a health condition in an animal 12, 22 (e.g., detect an estrus and/or off-feed condition in an animal 12, 22), establish a profile and/or baseline characteristics for an animal 12, 22 or group of animals, or the like.

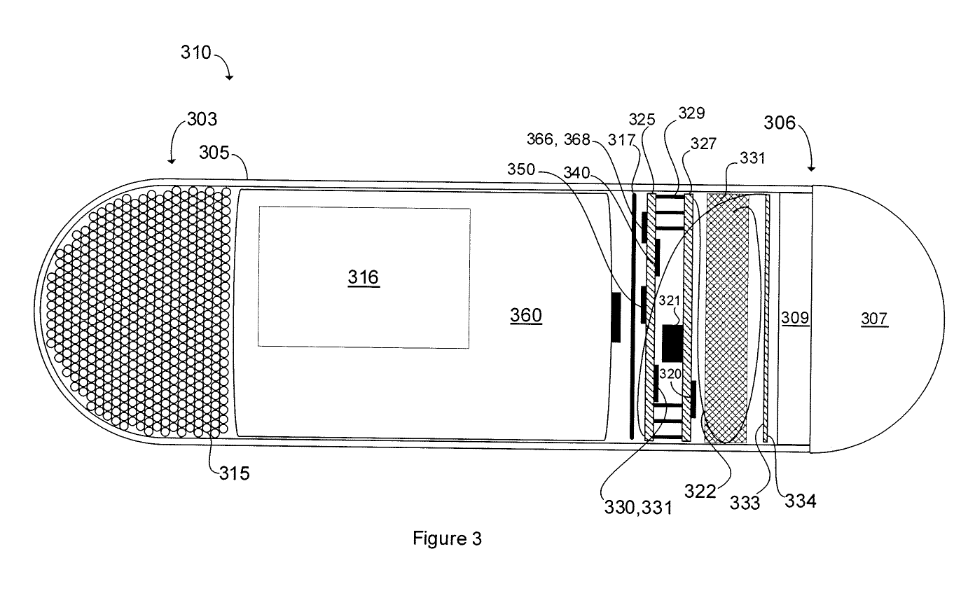

Turning now to FIG. 3, one embodiment of an ingestible bolus 310 is depicted. Bolus 310 may comprise a substantially hollow enclosure shell 305. A portion of the enclosure shell 305 may be substantially cylindrically shaped. As discussed above, enclosure shell 305 may be formed from any material capable of withstanding the environment within a stomach of a ruminant animal. In addition, the material of enclosure shell 305 may be formed from a non-toxic material such that it may not significantly adversely affect a host animal while disposed within the animal's stomach.

Enclosure shell 305 may be open at shell end 306. Cap 307 may be configured to fit within the open end 306 of shell 305. Cap 307 may comprise a feature 309 configured to securely engage an inner wall of shell 305 at end 306. As such, cap 307 may be securely press-fit onto shell 305. In an alternative embodiment, cap 307 may be secured onto shell 305 using a friction fit mechanism, a screw fit mechanism (e.g., with threads disposed on an inner surface of shell 305 and insert 309), adhesive, plastic welding, or the like. Cap 307 may be removable to allow the components disposed within enclosure 305 to be accessed.

The end 303 of shell 305 may be enclosed. As such, ballast weight 315 may be disposed therein. In the FIG. 3 embodiment, ballast weight 315 may comprise a plurality of substantially spherical weights (e.g., bearings, BBs, or the like). Alternatively, ballast weight 315 may be comprised of a formed solid material. Ballast weight 315 may be configured such that when bolus 310 is disposed within a stomach of an animal, bolus 310 may be maintained substantially in a bottom portion of the stomach. In this embodiment, bolus 310 may be maintained in contact with the portion of the stomach wall. As such, bolus 310 may be movably coupled to the stomach wall to allow an accelerometer sensor (e.g., element 321 discussed below) of bolus 310 to detect a contraction or other movement of the stomach and/or stomach wall. Since bolus 310 may be configured to contact a lower portion of the stomach wall, ballast weight 315 may be configured to cause bolus 310 to have a greater density than the fluids and/or feed commonly within the stomach of a host animal. Alternatively, bolus 310 may be movably coupled to the animal's stomach substantially as described above in conjunction with FIG. 2.

In some embodiments, ballast weight 315 may be comprised of a metallic material (e.g., iron, steel, or the like), which may be magnetized. In this embodiment, ballast weight 315 may attract magnetically active materials within the stomach of a host animal. In this way, bolus 310 may act as a cow magnet to prevent potentially hazardous objects (e.g., nails, tacks, etc.) from passing through the digestive system of a host animal.

Bolus 310 may comprise message label 316. Message label 316 may indicate that if found, bolus 310 should be returned to the animal manager and/or manufacturer of the bolus 310. As such, label 316 may comprise a mailing address. Label 316 may be disposed on an outer portion of enclosure shell 305 to be visible thereon. Alternatively, if enclosure shell 305 is comprised of a substantially transparent material, label 316 may be disposed on an inner portion of enclosure shell 305. This may prevent label 316 from being degraded by acid and/or abrasion when bolus 310 is disposed within a stomach of an animal. Alternatively, label 316 may be formed into or onto enclosure shell 305 during manufacture (e.g., etched and/or embedded into enclosure shell 305). When returned to the animal manager and/or manufacturer, bolus 310 may be refurbished, upgraded, disposed of, and/or redeployed.

A power source 360 may be disposed within shell 305 of bolus 310. As discussed above in conjunction with FIG. 2, bolus 310 may comprise a power source 360 to power the electrical components thereof (e.g., sensors 320, communication module 330, processor 340, memory unit 350, and the like). Power source 360 may comprise energy storage means including, but not limited to: a lithium ion battery; a lithium polymer battery; a lead acid battery; a nickel cadmium battery, an alkaline battery, a capacitor, a high-capacity capacitor, a super capacitor, or the like. Power source 360 may comprise a power generation means, such as a piezoelectric generator, a heat energy generator, an MFC generator, a movement and/or motion activity generator, or the like. Power source 360 may comprise a combination of energy storage means and energy generation means to generate and/or provide power to bolus 310. One skilled in the art would recognize that any power source could be used under the teachings of this disclosure. As such, this disclosure should not be read as limited to any particular power source 360 implementation.

In the FIG. 3 embodiment, power source 360 may comprise a substantially cylindrical battery energy storage device 360 having a diameter configured to securely fit within an inner wall of enclosure shell 305.

Bolus 310 may comprise an insulator 317 disposed between power source 360 and first and second printed circuit boards (PCB) 325, 327. Although FIG. 3 depicts 325 and 327 as PCBs, one skilled in the art would recognize that any circuit substrate could be used under the teachings of this disclosure. As such, this disclosure should not be read as limited to any particular circuit substrate material. As used herein, a substrate may refer to any supporting material on which a circuit is formed or fabricated. Insulator 317 may protect the circuitry disposed on first PCB 325 from power source 360 and/or may prevent unwanted electrical communication between the circuitry of PCBs 325 and 327 and energy storage 360 (e.g., prevent short circuiting, or the like).

Power source 360 may be electrically coupled to the electrical components of bolus 310 via voltage regulator 368 and power monitor 366. As discussed above, power monitor 366 may be configured to monitor a power and/or voltage level of power storage 360 to thereby detect a low-power condition in power storage 360. Power level information obtained by power monitor 366 may be provided to processor 340 and/or communications unit 330. Voltage regulator 368 may be configured to provide a constant voltage input to the electrical components of bolus 310 despite fluctuations in the voltage level of power storage 360.

The electrical components of bolus 310 may be disposed on a first PCB 325 and a second PCB 327. In embodiments employing fewer and/or smaller sized circuitry components, bolus 310 may comprise a single PCB. First and second PCBs 325 and 327 may be substantially flat and substantially disk-shaped. The diameter of the first and second PCBs 325, 327 disks may be configured to allow first and second PCBs 325 and 327 to fit within an inner wall of enclosure shell 305.

First PCB 325 may be joined to second PCB 327 using prongs 329. Prongs 329 may be disposed along an outer diameter of first and second PCBs 325 and 327 to join first PCB 325 to second PCB 327. One or more prongs 329 may act as a via to provide electrical communication between circuitry disposed on first and second PCBs 325 and 327.

The electrical components of bolus 310 may be disposed on first and second PCBs 325 and 327 and electrical communication therebetween may be provided by one or more PCB traces disposed on PCBs 325 and 327 and/or prongs 329. The electrical components may comprise one or more sensors 320, including an accelerometer 321 and thermistor 322. Accelerometer 321 may be a three-axis accelerometer. Thermistor 322 may be configured to detect an internal temperature (e.g., stomach temperature) of an animal. The electrical components may further comprise a communications module 330, a processor 340, and a memory unit 350. Communications module 330 may comprise a transceiver 331. Transceiver 331 may be capable of transmitting and receiving data wirelessly using antenna 333.

As discussed above, although not shown in FIG. 3, bolus 310 may comprise an electrical connection between power source 360 and first and/or second PCBs 325 and 327 to provide power to the circuitry disposed thereon (e.g., sensors 320 and 321, communications module 330, processor 340, memory unit 350, and the like). The power connection (not shown) may pass through voltage regulator 368 to regulate an input voltage to the electrical components of bolus 310. In addition, a power monitor 366 may monitor a power and/or voltage level in power supply 360.

A substantially disk-shaped insulator and/or cushioning member 331 may be disposed between first and second PCBs 325, 327 and transmitter antenna PCB 334. Insulating cushion 331 may provide cushioning between first and second PCBs 325, 327 and antenna 333. In addition, cushion 331 may act as a spacer to separate antenna 333 from the circuitry disposed on first and second PCBs 325 and 327. This spacing may reduce radio frequency (RF) interference and/or capacitive coupling between the circuitry on PCBs 325 and 327 and antenna 333.

Antenna 333 may be disposed on antenna PCB 334. As such, antenna 333 may be comprised of one or more traces on antenna PCB 334. This may allow antenna 333 to be precisely positioned on antenna PCB 334. In addition, forming antenna 333 as a trace on antenna PCB 334 may ensure that antenna 333 does not deform or otherwise change its orientation within enclosure shell 305 due to movement of bolus 310 within an animal. Antenna 333 may be used by communication unit 330 and transceiver 331 to both transmit and receive data wirelessly (e.g., on an RF carrier).

Bolus 310 may further comprise padding (not shown) disposed between antenna PCB 334 and cap 307 and cap insert 309. Such padding (not shown) may be used to secure components 315, 317, 325, 327, 331, 334, and 360 within bolus enclosure shell 305. In some embodiments, cap 307 and cap insert 309 may be hollow. In this case, padding (not shown) may be used to fill the hollow areas of cap 307 and cap insert 309 to secure bolus 310 components 315, 317, 325, 327, 331, 334, and 360 within bolus enclosure shell 305.

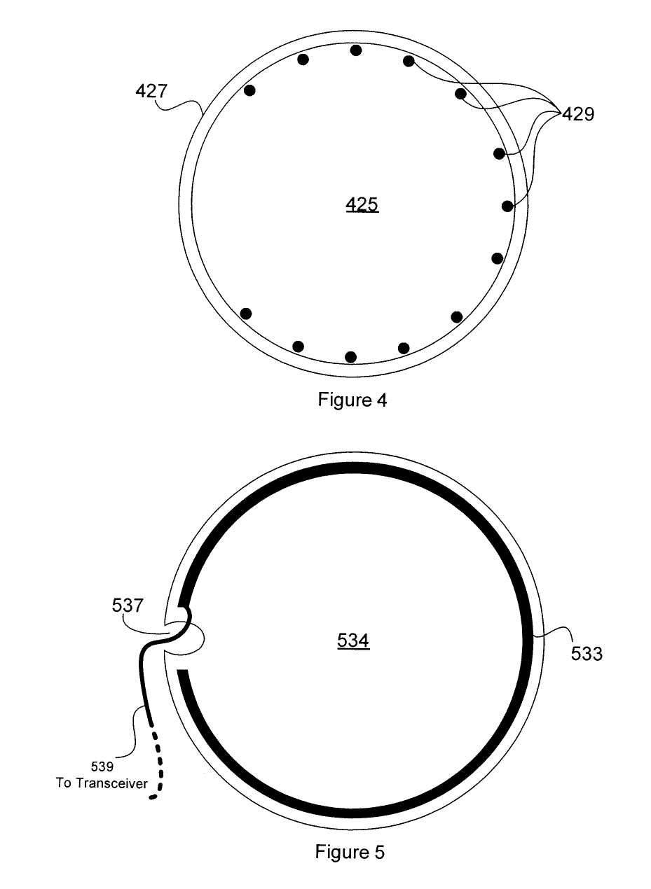

Turning now to FIG. 4, one embodiment of a first PCB 425 and second PCB 427 is depicted. First PCB 425 and second PCB 427 may be substantially disk shaped. The diameter of first PCB 425 and second PC 427 may be configured to allow the PCBs 425 and 427 to fit within an inner wall of a bolus enclosure shell, such as element 305 of FIG. 3. First PCB 425 may be jointed to second PCB 427 using prongs 429. Prongs 429 may be disposed on an outer portion of first and second PCBs 425, 427. Some of prongs 429 may provide electrical communication between components (not shown) disposed on first PCB 425 and components (not shown) disposed on second PCB 427.

Turning now to FIG. 5, one embodiment of an antenna PCB 534 is depicted. Antenna PCB 534 may comprise a notch 537 to allow a conductor 539 to contact antenna 533. Notch 537 may be used where the diameter of antenna PCB 534 is substantially equivalent to an inner diameter of a bolus shell (not shown). In this case, conductor 539 may only be capable of reaching antenna PCB 534 through notch 537. Antenna 533 may be comprised of one or more traces disposed on one or more layers of antenna PCB 534. Although FIG. 5 depicts antenna 533 as a single trace, one skilled in the art would recognize that there are numerous antenna configurations that could be traced onto antenna PCB 534. As such, this disclosure should not be read as limited to any particular antenna 533 configuration and/or orientation.

It will be obvious to those having skill in the art that many changes may be made to the details of the above-described embodiments without departing from the underlying principles of the invention. The scope of the present invention should, therefore, be determined only by the following claims.

I claim:

1. An ingestible bolus disposable within a stomach of an animal to measure one or more characteristics of the animal, comprising:
a sensor to measure an animal characteristic;
a transmitter communicatively coupled to the sensor to transmit the measured animal characteristic to a receiver;
a power source in electrical communication with the sensor and the transmitter to power the sensor and the transmitter; and
ballast weight configured to cause the bolus to be maintained in a lower portion of the stomach.

2. The ingestible bolus of claim 1, wherein the ballast weight is magnetized.

3. The ingestible bolus of claim 1, wherein the ballast weight is configured to cause the bolus to be movably coupled to a portion of a stomach wall of the animal.

4. The ingestible bolus of claim 3, wherein the sensor is an accelerometer.

5. The ingestible bolus of claim 4, wherein the accelerometer is a three-axis accelerometer configured to detect a contraction of the stomach.

6. The ingestible bolus of claim 4, further comprising a memory unit having bolus configuration data and an animal identifier stored thereon, wherein the transmitter is configured to transmit the animal identifier with the measured animal characteristic.

7. The ingestible bolus of claim 6, wherein the transmitter comprises a transceiver capable of receiving bolus configuration data for storage on the memory unit.

8. The ingestible bolus of claim 1, further comprising a substantially cylindrical enclosure shell to contain the sensor, the transmitter, the power source, and the ballast weight.

9. The ingestible bolus of claim 8, further comprising a message label disposed on an inner wall of the enclosure shell, wherein the enclosure shell is substantially transparent, and wherein the label provides a return address for the bolus.

10. The ingestible bolus of claim 8, further comprising a first, substantially disk-shaped circuit substrate, wherein a diameter of the first circuit substrate is configured to allow the first circuit substrate to be received within the enclosure shell, and wherein the sensor and the transmitter are disposed on the first circuit substrate.

11. The ingestible bolus of claim 8, further comprising:
a substantially disk shaped antenna substrate having a diameter configured to allow the antenna substrate to be received within the enclosure shell; and
an antenna formed as a trace on the antenna substrate, wherein the antenna is communicatively coupled to the transmitter.

12. The ingestible bolus of claim 10, further comprising an insulating spacer to separate the antenna substrate from the sensor, the transmitter, and the processor.

13. The ingestible bolus of claim 1, wherein the power supply a generator.

14. An ingestible bolus disposable within a stomach of an animal to measure one or more characteristics of the animal, comprising:
a three-axis accelerometer;
a transmitter communicatively coupled to the three-axis accelerometer, wherein the transmitter is configured to transmit measurements derived from the three-axis accelerometer to a receiver;
a power supply electrically coupled to the three-axis accelerometer and the transmitter to power the three-axis accelerometer and the transmitter; and
an enclosure shell,
wherein the three-axis accelerometer, the transmitter, and the power supply are disposed within the enclosure shell.

15. The ingestible bolus of claim 14, further comprising ballast weight disposed within the enclosure shell, wherein the ballast weight is configured to cause the bolus to be maintained in movable communication with a portion of a stomach wall of the animal.

16. The ingestible bolus of claim 14, wherein a portion of the enclosure shell is substantially cylindrical, the ingestible bolus further comprising:
a processor communicatively coupled to the three-axis accelerometer and the transmitter; and
a memory unit communicatively coupled to the processor,
wherein the memory unit comprises instructions to cause the processor to control the three-axis accelerometer and the transmitter, and
wherein the accelerometer, the transmitter, the processor, and the memory unit are disposed on a substantially disk-shaped circuit substrate disposed within the enclosure shell.

17. The ingestible bolus of claim 16, further comprising:
a substantially disk-shaped antenna substrate disposed within the enclosure shell; and
an antenna communicatively coupled to the transmitter formed as a trace on the antenna substrate

18. The ingestible bolus of claim 17, further comprising an insulating spacer to separate the antenna from the accelerometer, the transmitter, the processor, and the memory unit.

19. The ingestible bolus of claim 14, further comprising a temperature sensor communicatively coupled to the transmitter disposed within the enclosure shell, wherein the transmitter is configured to transmit measurements derived from the temperature sensor to a receiver.

20. A method for monitoring an animal characteristic, the method comprising:
placing an ingestible bolus within a stomach of a ruminant animal, the ingestible bolus comprising a ballast weight configured to cause the bolus to be maintained movable communication with a portion of a stomach wall of the animal;
measuring an acceleration characteristic of the animal using an accelerometer disposed within the bolus; and
transmitting the measured acceleration characteristic using a radio frequency transmitter disposed within the bolus.

21. The method of claim 20, wherein the acceleration characteristic is a vector magnitude of a three-axis acceleration measured by the accelerometer.

22. The method of claim 20, wherein the acceleration characteristic is a time derivative of a vector magnitude of a three-axis acceleration measured by the accelerometer.

23. The method of claim 20, wherein the acceleration characteristic corresponds to animal stomach contraction activity.

24. The method of claim 20, wherein the measured acceleration characteristic corresponds to animal movement activity.

25. The method of claim 20, wherein the measured acceleration characteristic corresponds to animal stomach contraction activity and to animal movement activity.
